(19) United States
(12) Patent Application Publication
(10) Pub. No.: US 2005/0130320 A1
(43) Pub. Date: Jun. 16, 2005

(54) Method for identifying the proteome of cells using an antibody library microarray

(76) Inventors: Shaji. T George, New York, NY (US); Roberto Burioni, Rimini (IT)

Correspondence Address: Christine M Rebman, P O Box 2828, Chicago, IL, 60690-2828, US

(21) Appl. No.: 10/416,435

(22) PCT Filed: Nov. 8, 2001
(86) PCT No.: PCT/US01/46753

Related U.S. Application Data
(60) Provisional application No. 60/247,312, filed on Nov. 9, 2000.

(51) Int. Cl. G01N 33/00; C12P 21/00; C12N 5/16; G01N 33/543; C07K 16/18

(52) U.S. Cl. 436/518; 800/6; 435/326; 530/387.1

(57) ABSTRACT

The invention describes a method for identifying the level of various proteins in a cells or tissue and also comparing the levels of the same protein in two different tissues which we have termed as Antibody Microarray Proteomics Technology (AMP Technology). Proteins that are differentially expressed between a normal and disease tissue could be involved in the disease process and disease tissue could be involved in the disease process and hereby makes it a potential drug or diagnostic target. AMP technology makes use of the method of phage display selection or similar combinatorial antibody selection techniques to select antibodies to a given protein. Antibodies to all of most proteins present in the diseased and normal tissue are produced in an appropriate animal (mice) and mRNA encoding antibodies are isolated, cloned into filamentous phage (or bacteria or yeast) vectors such that the antibodies are expressed as a fusion with the phage filaments. Phage clones that express antibodies to different proteins in a tissue are microarrayed on a glass slide coated with polystyrene using an automated array spotter. Proteins from both the disease and normal tissues are extracted, conjugated with a flourescent label and incubated on two seperate but identical arrays. Once the proteins are bounded to the antibodies on the array, they are washed and scanned using a flourescent scanner, which will quantitatively determine the level of each protein present in the cell.

### RELATED CASES

This application claims priority to U.S. patent application Ser. No. 60/247,312 filed on 9 Nov. 2000, which is incorporated herein by reference in its entirety.

### FIELD OF THE INVENTION

The present invention is related to a method for using phage display immunoselection or similar techniques in order to use combinatorially selected antibody or antibody fragments against cellular proteins so as to allow one to quantitatively and qualitatively determine the level of known and unknown proteins in a cell or tissue by arraying a sufficiently large number of such antibodies to allow maximum representation of antibodies against the cellular proteins and capturing the cellular proteins from the cells or tissues of interest. The method allows one to compare the levels of a known or unknown protein between a normal and a disease tissue or a cell as well as allow to determine the level of various known and unknown proteins in a cell or tissue.

### BACKGROUND

Proteomics is the study of the proteome (all the proteins produced from all the genes of a genome). Proteomics is an emerging field in biomedical research and is believed by many to become the new century's superhighway to biomedical knowledge. Proteins carry out most functions of the human body. When levels of certain crucial proteins are altered or when they are mutated or when their interaction with other proteins is disrupted, it often leads to disease. Understanding such changes in protein levels and function is crucial for proper understanding of the disease process, which in turn will spur the development of a host of new drugs and diagnostics to effectively manage these diseases. Proteomics deals with understanding the role of proteins in health and disease. Already a dramatic shift in the emphasis from DNA and mRNA (which codes for proteins) sequencing (exemplified by the several genomic companies) to the study of proteins involved in diseases is becoming evident. It is dawning on government-funded laboratories and drug companies that mass sequencing of genomic DNA and spotting cDNA onto chips may not lead to the promised land.

Limitations of Genome-Based Drug Target Identification Techniques:

The genomic revolution will undoubtedly generate massive amount of important information on the genes. However, a far greater challenge is to determine the function of each of these genes and their role in the etiology and progression of human diseases. Only through such knowledge can the vast amount of genomic information gathered can be put to practical use. Much of the deciphering of the genomic information will come from the understanding of the function of the proteins encoded by these genes and the field of proteomics will play a very major role in this process.

The genomic technologies despite their high profile, have major shortcomings. They do not take into account:

pre-translational events (mRNAs coding for various proteins being cut and spliced to which leads to their translation into variants such proteins with different functions), translational efficiency (the number of protein molecules that can be made from one copy mRNA, which varies widely from mRNA to mRNA) and

Post-translational modifications (most often it is not the level of proteins, but modifications like posphorylation that modulate the function of proteins).

There are no drugs that are mRNAs and there are only a handful of drug targets that are mRNAs, but proteins are and it is essential to understand the role of proteins in diseases to develop new drugs and diagnostics.

Knowledge about the role of various proteins in diseases (like the role of NEM in prostate cancer) will be invaluable to major drug and diagnostic companies and they are investing heavily for obtaining the commercial rights to develop products based on such targets. Most experts in the filed believe that unraveling the complex proteome of the cell will revolutionize the biomedical field and lead to the discovery of a new generation of drugs and diagnostics.

Current Techniques of Proteome Analysis and Their Limitations:

Proteomics technology is still in the process of being defined. Currently there are a dozen companies and several academic institutions involved in preotomic research.

A promising technology that is being developed by Phylos (Cambridge, Mass.) uses a new technology called, mRNA display, which uses covalent fusion between an mRNA present in a cell and the protein encoded by such mRNA. The complex can then be separated on a DNA chip. The technique relies on isolating mRNA from tissues in the state in which is present in a functioning cell followed translating it in-vitro without any alteration in the level of mRNA due to degradation, a tall order due to the rapid breakdown of mRNA in cell extracts. Even if this hurdle can be overcome, the translational efficiency of mRNA within a cell may be significantly different from an in-vitro system, thus skewing the results. The technology has several other technical hurdles to overcome. Another company called Ciperagen (San Diego, Calif.) manufactures small chips, which is coated with media that can separate proteins. Separated proteins can then be analyzed by MALDI-TOF. However, it is difficult to isolate proteins from such media for further characterization. Other companies like DYAX and Cambridge Antibody Technology are attempting to generate antibodies against proteins that are identified by the genome sequencing effort (currently protein sequence encoded by 30,000 of the more than 100000 genes are known) and using such antibodies to separate proteins. These technologies don't take into account many of the modifications that are crucial in determining the role of proteins in diseases.

To achieve the oft-stated goal of proteomic analysis requires that one be able to separate all the proteins present in the cell, identify them, and analyze the state of their post-translational modifications. “Global proteomics” of this sort is often likened to the use of array technology to scan genes for mutations or to sample mRNA population of a cell. It is important to note that this is a facile comparison and proteomics is considerably more complicated than functional genomics. The latter techniques do not require separation of the mRNA population into its individual components. However, one must keep in mind that because of alternate splicing and post-translational modifications, there are far more protein moieties in the cell than there are genes. Thus, true global proteomics may require techniques that can resolve as many (or more than) twenty thousand proteins—certainly at least ten thousand—from any given cell or tissue type and identify these proteins successfully in the face of a likely 100,000-fold variation in levels of abundance.

Currently, in academic and industrial settings, centers for proteomics almost invariably follow the same model: use of a two-dimensional (2D) gel electrophoretic analysis to separate proteins and protein mass fingerprinting by mass spectrometry or chemical sequencing of peptide fragments in order to identify the proteins present in the spots on the gels.

The main limitation of this technique is that the gels rarely resolve more than 1000-2500 proteins present in a cell while there are 15000-20000 or more different protein species present in any given cell. Furthermore the major proteins present in a cell that have less role in disease process (structural proteins) tend to mask the less-abundant proteins that have far more crucial role in diseases. Despite the fact that even the best protein microchemisty facilities can identify, under the most favorable circumstances, perhaps 10-20 proteins a day, the marriage of these techniques has often been termed as “high throughput proteomics”. It is indeed very difficult to automate the 2D-gel technique and it is inherently a slow process.

In order to identify proteins involved in disease, the proteins from normal and disease tissue are extracted and separated on 2D gels, stained with a suitable dye and visualized. Proteins whose abundance varies significantly between the normal and disease tissue are targeted for further study. The protein spots are manually cut out from the gel (a slow and tedious process), the proteins eluted from the gel, and the identity of these proteins determined by peptide fingerprinting using MALDI-TOF (Matrix Assisted Laser Desorption Time of Flight) mass spectrometry or by chemical sequencing. Attempts at using MALDI-TOF spectrometry to determine the sequence without eluting the proteins from the gel are underway, but not yet perfected.

Requirements for True High Throughput Proteomics:

Reaching the stated goal of high throughput “global” proteomics will therefore demand significant new technologies for:

efficient and rapid separation of all proteins present in a cell or tissue

identification of even minor proteins present in a cell, which may be 100,000-fold less than the most abundant protein

techniques to identify the separated proteins in the separation media or techniques that can rapidly identify the proteins outside the media

automation of the process

### SUMMARY OF THE INVENTION

Disclosed is a method and composition for the study of the proteome of a cell or tissue. The invention called high-throughput “Antibody Microarray Proteomic Technology” (AMP technology) would overcome many of the inherent deficiencies associated with the current proteomics methods. The key to the technology is the ability to rapidly develop a library of monoclonal antibodies directed against most of the proteins present in a cell. The antibodies are then arrayed on a microarray slide and the various proteins present in disease and normal tissues are rapidly separated on the microarray. Proteins whose levels are significantly altered or proteins that are modified in a disease tissue could be potential drug targets. The partial sequence of these proteins can then be easily determined using currently available mass spectrometric techniques (MALDI-TOF) and automated chemical sequencing. Such proteins can be further purified in larger quantities relatively easily using the same antibody. Proteins of interest are further analyzed in detail for their role in disease process. The technology would be significant improvement over currently used proteomic techniques for the identification of potential protein drug targets. Furthermore, the same antibody that was used for the identification of the potential target can be used for further validation of the target in cell culture and animal models thereby expediting the process of drug discovery and saving time and resources. Even intracellular proteins can be studied by cloning the cDNA encoding the antibody into each cell to produce specific knockouts, a tremendous advantage over other techniques. The key aspects of this proprietary technology are outlined in figure Specifically disclosed are methods for production of antibodies against most proteins including many proteins present in smaller amount antibodies in animals, preventing the dominance of certain major epitopes over minor ones. The invention further discloses displaying the antibody fragments on a filamentous phage followed by selecting the phage expressing the antibodies against the proteins and arraying phage clones displaying individual antibodies against each protein on a glass slide and using such microarry to capture proteins in a cell or tissue that are prelabeled with a detection tag like flourescein and monitoring the level of binding of the protein to each antibody on the array using a fluorescent scanner. In the preferred embodiment, one would array a very large number of antibodies or phage displaying antibodies on the slide (usually exceeding 0.5 million) in such a way that there is maximal representation on the array of the various antibodies against different proteins.

Also disclosed is the use of such antibody microarrays to determine the level of various known and unknown proteins that are present in diseased and normal cells. By using identical arrays to capture proteins present in a disease and normal tissue, one can determine the level of each protein that is present in each of the tissue by determining the amount of fluorescent-labeled protein bound to the antibody. Identification of the levels of each protein would allow one to determine the potential role of each protein in the disease process. Such proteins could be potential targets for therapeutic intervention or could potentially be used as markers for diagnosis of diseases.

Proteins of interest (proteins that are differentially expressed between disease and normal tissue) are purified in larger amount by immunoaffinity chromatography using the same antibody

The invention has several advantages over some of the current technology, which uses two-dimensional gel electrophoresis as described below.

the technology allows rapid (high throughput) analysis of thousands of proteins present in a tissue simultaneously

The arraying and screening can be automated, allowing thousands of samples to be analyzed each day

Antibodies against proteins present in a tissue are produced simultaneously using techniques developed in our laboratory

Unlike 2-D gel separation techniques (which allows separation of only 5-10% of the proteins in a tissue), AMP technology allows the separation of almost all the proteins present in a tissue

No messy cutting out gel pieces or elution from gel, a tedious and inefficient process

2-D gel based proteomic techniques require the fragmentation of proteins prior to their elution from gel. Hence using 2-D gel techniques one cannot usually obtain intact proteins, but only fragments but ANT technology allows the isolation of full-length proteins enabling more precise characterization of the protein.

The technique should allow the detection of even proteins that are expressed in very small amounts

The technique allows the detection of even many proteins that are post-translationally modified, particularly when they are phosphorylated or sulfated, key modifications that alter protein function

The same antibody in the array that was used for the capture of the protein can be used for immediate larger scale purification of the protein by antibody affinity chromatography for further characterization.

Another very important feature of AMP technology is that once a differentially-expressed protein target has been identified, the same monoclonal antibody that was used in for identification (and isolation) of the protein target can be used for further easy validation of many target proteins (similar to how NEM antibody was used to identify the role of NEM in cancer)

If the target were intracellular, then the cDNA encoding the antibody, which can be easily isolated from the phage using simple PCR techniques, would be cloned into immortalized cells from the disease tissue. The intracellulary-expressed antibodies can be used as molecular knockouts of target proteins thereby helping to further understand the role of these proteins in the disease process. This will considerably accelerate down stream target validation, a major advantage over contemporary methods.

### BRIEF DESCRIPTION OF THE DRAWINGS

FIG. 1 describes a schematic of the entire process of Antibody Microarray Proteomics Technology

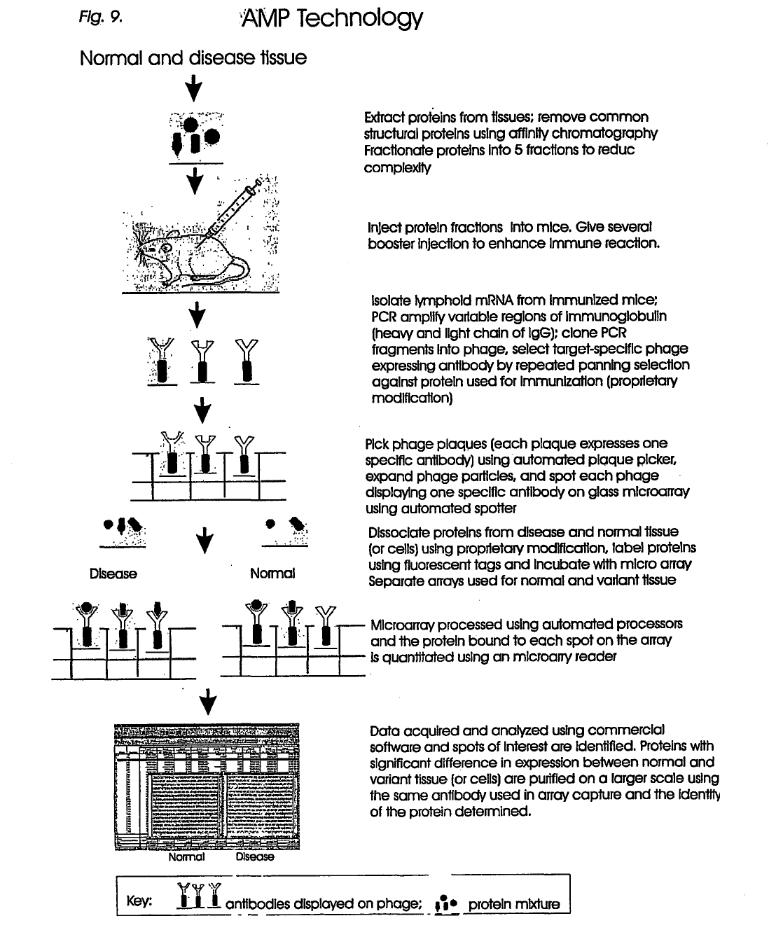

### DETAILED DESCRIPTION OF THE INVENTION

A method of, and composition for, generating antibodies against many or all proteins present in a cell using combinatorial antibody selection in mice or other animals is described. Also described is the use of such a library of antibodies arrayed on a microarray slide and use of such microarray to separate fluorescent-tagged proteins that are present in a cell. The fluorescent-tagged proteins are then captured by the antibodies on the microarray and quantitated by measuring the level of fluorescence bound to each spot on the array using a fluorescent scanner.

Extraction of Proteins from Cells:

A variety of published methods are available for extracting intact proteins from biological materials like cells, tissues or pathogens like bacteria or fungi or plant materials. If the biological material is mammalian cells, the cells are generally disrupted using a Dounce homogenizer or a Potter-Elvejhem motor driven homogenizer in a buffer containing isotonic salt solution at near neutral pH and detergents like Chaps, Triton X-100, NP 40 or other detergents which are usually non-ionic, but may be anionic or cationic. Generally such extraction buffers should contain protease inhibitors like phenyl methyl sulfonyl fluoride or peptides like leupeptin or aprotinin. Furthermore, the biological material should be as fresh as possible or should be freshly frozen to prevent degradation of proteins. If one does not intend to extract membrane proteins, it is not necessary to use detergents. After the extraction, the debris is separated by centrifugation and the supernatant is used for further steps. If there is a layer of fat (as with liver tissues) that is also removed. Another agent that dissolved proteins effectively is 0.1 N NaOH, which can be neutralized easily with an acid like trichloroacetic acid or dichloroacetic or Tris base acid after extraction. This will obviate the need to use detergents. Often it is advisable to remove the detergents before they are used to prevent micelles trapping multiple proteins within them. For example, one could precipitate the extracted proteins with tricholoroacetic acid, washed several times with the same acid, and the precipitate washed with the same acid and then dissolved in 0.1 N NaOH and the solution neutralized with a buffer like Tris base. Alternatively certain commercially available organic solvents can be sued remove detergents. However, one has to be careful to add back any small peptides that are not precipitated with acid. Several other diverse methods have been used to extract proteins and the above methods are only some commonly used examples. Often disruption with an ultrasonic sound (sonicator) also helps to break up the membranes and other organelles and release proteins. This method increases the efficiency of solubilization by detergents or other agents.

Even in the absence of detergents some proteins tend to bind or stick to one another proteins through Van Der Waals forces or hydrogen bonds or ionic bonds or other types of hydrophobic or hydrophobic or hydrophilic interactions. Many hydrophobic interactions can be disrupted by mild chaotropic agents like KSCN while hydrophilic or ionic interactions can often be disrupted by salt. Covalent disulfide bonds can be broken by treatment with beta-mercaptoethanol.

However, if one needs to achieve complete separation of proteins, one can treat the protein mixture with a mild protease like stromolysin, chymotypsin, cathepsin D etc. This will release hydrophilic peptides, which does not stick to each other in mild salt solutions (0.15 M NaCl). The hydrophobic peptides that may clump (only some peptides will tend to clump) can be removed by centrifugation or hydrophobic chromatography on a C-3 or C-4 column or phenyl Sepharose chromatography or sometimes even by ultracentrifugation. Subjecting complexes to ultrasonic sound (sonicator) also helps to break up potential aggregates.

Source of proteins may include human tissues or cells, other animal tissues, pathogens like bacteria, fungi, parasites like malarial parasite, or plants depending on the proteome one wants to study.

Immunization of Host:

Different types of animals can be used for immunization with the proteins. Although the preferred animal is the Balb C mouse, other animals like rabbits, rat, or larger animals can be used. Prior to immunizing the animals, the biological material is homogenized very finely with either a Polytron homogenizer or a motor driven homogenizer in buffer or mild solublizing agents that are not toxic to the animals. The extract can be directly injected into the animals without further separation.

However, in order to obtain the best results, one should remove the major structural proteins that are present in the cell like histones, ribosomal proteins, albumin, certain cytoskeletal proteins etc. Such proteins make up more than 90% of the protein mass of the cells. They can be easily removed by immunoaffinity chromatography using a mixed immunoaffinity resin. Such an inmmunoaffinity resin will have antibodies against the structural proteins coupled to it. The protein mixture prior to immunization can be removed by binding them to the antibodies immobilized on the resin. The protein that does not bind to the resin (non-structural proteins) can be then used for immunization of mice. Several methods of immobilizing antibodies to various matrices like Sepharose, Sephacryl etc., have been described by many authors. Antibodies to most major structural proteins are commercially available.

To further improve the chances of generating antibody against even minor proteins, one should extract the proteins using one of the methods described above. It is not always necessary to completely break up aggregates since they will be proteolytically degraded by the mouse.

Prior to immunization, it is useful, but not always necessary to separate the proteins into multiple fractions (5-50). Each fraction can then be injected into a separate mouse. This will allow one to decrease the number of diverse proteins that are injected into each animal and will increase the chance that there is adequate representation of even minor proteins in sufficient amounts injected into each animal. This also will allow larger amount of such minor or poorly antigenic proteins to be injected into each animal. The fractionation of the extracted proteins into various fractions can be performed using chromatographic techniques. These techniques include, but are not limited to ion exchange chromatography on an anion or cation exchange resin, gel filtration, electrophoresis, electrofocussing, hydrophobic chromatography or other protein separation techniques. Several methods of separating proteins into fractions are published. The separated proteins are collected in fractions and each fraction is separately injected into different animals.

Preferred routes of injection include injection into the popoteal lymph nodes or subcutaneous injection. It is necessary to use an adjuvant like RIBI immunogen or similar products to enhance the immune response. Booster doses of the protein are injected after one and two-week interval and the animals sacrificed after 30 days or more, the spleen of the animals are removed, and mRNA extracted as described above. If the protein mixture were injected in multiple mice, then the mRNA fractions are pooled before further processing. The variable chain of the antibody chains (V_{H} and L_{H}) encoding the immunoglobulins is amplified using IgG specific primers using PCR techniques. The PCR amplified fragments are cloned into the phage vector using standard recombinant DNA techniques.

Alternatively, one could immunize mouse or other transgenic animals that carry genes that express human antibodies. This will generate human antibodies, the cDNA of which can then be cloned into appropriate vectors.

Cloning of Antibody cDNA into Vector:

One can follow several methods for cloning the antibody cDNA into various vectors. A common method of displaying antibodies is by cloning the cDNA encoding the antibodies into the gene encoding the filament of filamentous phages (Mc Cafferty et al 1990; Barbas, et al, 1991). In general antibodies or the fragments of antibodies (variable regions) have been fused to the N-terminus of the minor coat protein pIII of the phage or to the C-terminal domain of pIII and displayed as Fab fragments (Hoogerboom, et al, 1991) or as single chain Fv (scFv) fragments (Mc Cafferty, et al, 1990) in which the CH and VL domains are linked by a flexible polypeptide (Bird et al., 1988; Huston et al, 1988). With Fab fragments, one chain is fused-to the phage and the other secreted into the cytoplasm. Alternate hosts like bacteria or other unicellular organisms can also be used for displaying antibody or fragments of antibody. Protease sensitive sites can be engineered at both termini of the antibody or fused V_{H} and L_{H} fragments so that they can be released as functional antigen binding sequences by treating with the appropriate proteases, which can then be separated, from the phage or bacterial host.

For cloning the antibody fragments in the appropriate vector, the total mRNA from the spleen or other source of lymphocytes or spleen of the immunized animal (or human bone marrow) is extracted taking care to prevent degradation using standard methods of mRNA isolation. Repertoires of antibody fragments are first generated by PCR from rearranged V genes (Orlandi et al., 1989; Ward et al,1989; Husc et al, 1989; Clackson et al 1991). Once could also use the peripheral blood lymphocytes or bone marrow (preferred) from humans, if one intends to generate an antibody array of antibodies isolated from humans (Williamson et al, 1993; Burioni et al, 1994). The latter technique is particularly useful for studying autoimmunity as well as pathogenic infection in human subjects.

A variety of vectors can be used for expressing antibodies on phage filaments. These include phage vectors, which encode the pIII fusion and all functions required for replication, packaging, and infection of bacteria, phagemid vectors, which require rescue with a helper phage. The latter vectors comprise the pill fusion, plasmid, and phage origins of replication and antibiotic resistance markers. Other vectors, fusing the antibody to other phage proteins, as protein VIII, more represented in the phage structure, could also be used for a full exploitation of the procedure. The helper phage provides the other functions for single-stranded replication and packaging. Helper phages are very poorly packaged in competition with phagemids due to defective origin.

Amplification of Library:

The bacterial strains TG1[K12, D(lac-pro), supE, thi, hsdD5/F′traD36, proA+B+, lacI, lacZDM15] are typically used for propagation of phage particles and HB21521 nonsupressor strain (K12, ara, D(lac-pro), thi/F″ proA+B+, lac^{q}ZDM15) for expression of antibody fragments. If a phagemid is used for cloning the antibody fragments, a helper phage like VCS M13 (Stratagene) and M13 K07 (Pharmacia) can be used to rescue the phage displaying the antibody fragments as described by the manufacturer.

The phage library in the appropriate bacterial host is grown and harvested in log phase (0.6 OD). The phage particles are isolated by standard methods (Harrison, et al 1983).

The antibody cDNA that are represented in the library at this point will have all the antibodies expressed by the host and not merely the antibody to the antigens that were injected. Furthermore, the majority of combination of heavy and light chains will not generate fully functional Fabs. Hence the phage expressing the antibody to the injected targeted antigens are selected by panning against the same proteins that were injected. The antigens are usually immobilized on a polystyrene or similar support. The stringency of selection can be increased by reducing the density of coating of the antigen to the solid surfaces or by increasing the time of washing or the concentration of detergents. To avoid nonspecific binding of phage to the polystyrene or other surface, milk proteins or other commercially available blocking agents are used. Usually the proteins are suspended in PBS containing low concentration of detergent (0.1% Tween 20 or NP 40). The antigens that are immobilized are the same as that the ones that were injected into the host. Immobilization is usually achieved by overnight incubation of the antigens with the support (immunotube) overnight and unbound proteins removed by washing with PBS.

The phage particles (10¹²-10¹³ t.u) are bound to the immobilized protein in the immunotube, by incubating them in the immunotube for 30 minutes and washed a few times with PBS containing 0.1% Tween and with PBS alone and the phage the bound phage is eluted usually with 100 mM triethylmaine and immediately neutralized with Tris-HCl pH 7.4.

The eluted phage is used directly for plating on agar plates. However, if the too many phage particles expressing irrelevant antibody fragments or no antibody fragments at all are present, one or two more cycles of panning are performed. This is done by infecting bacteria with the eluted phage, amplifying the library and (after helper phage infection) the phage harvested. The harvested phage is again panned against the immunotube with the bound antigens, washed and the bound phage eluted. More than three cycles of panning is not advised since it reduces the representation of antibodies that are present in the library.

One major difference of the present technique from routine techniques that are used for isolating antibodies displayed on phage or bacteria is that fewer cycles of selection are employed. Generally they are limited to one or two cycles. This increases the representation of the antibodies against minor proteins or poorly antigenic proteins.

Competitive Deselection of Immunodominat Epitopes:

This technique is critical for the increased representation of antibodies against less represented antigenic determinants. It also increases the efficiently of antibodies of rare specificities. The inclusion of the technique allows far fewer antibodies to be arrayed on the microarray to represent antibodies against most of the antigens. The protocol is a modification of a standard panning protocol, except that the phage library is first preabsorbed on the antigen of interest to remove phage that react with the immunodominat epitope. The unbound phage are then incubated a second time with the antigen, and then eluted and amplified as per normal protocols. However, the subtraction of unwanted clones is only partial, and should be considered as “negative enrichment” rather than a complete substraction. Although subtracted clones are usually present at the end of the panning procedure, their frequency is considerably lower and that obtained with an unmodified panning procedure. This allows for the representation of rare clones and does not affect the success of the technique. The same technique can also be used if the vector is a bacterium.

Isolation of Phase Clones:

The phage library is used to infect a bacterial lawn such as to produce well-separated phage plaques (Sambrook et al, 1991) after helper phage infection. Generally 100 or more of 150-mm plates are necessary to generate a library of phage that would display antibodies against most of the antigens injected. If the antibody is displayed on a bacterial host having an antibiotic resistance gene, the bacteria is plated an antibiotic plate and the colonies harvested.

The phage clones are either harvested manually using toothpicks or using an automated plaque picker (like GeneTAC G³ from Genomic Solutions, Cambridge, Mass.). The plaques are used to infect bacterial host that are plated in multiwell plates and amplified. Aliquots of the phage are harvested, and used for arraying on glass or plastic microarrays. If the antibody is displayed on bacterial host, the bacterial colonies are grown in multiwell plates and aliquots are removed for arraying on microarrays.

Microarrying of Phase or Antibodies or Fab Fragments:

The phage clones or bacterial clones displaying different antibodies arrayed on a microarray slides similar to the microarry slides manufactured by Molecular Dynamics/Amersham Pharmacia Biotech. The slides are coated with polystyrene or similar materials that can bind proteins. Of particular use is the method used by Khrapko, et al (1996) for immobilization of oligonucleotides, which can be modified for protein coupling, or Versalinx (Prolinx, Bothell, Wash., USA) slides. The former uses polyacrylamide pads for coupling proteins. The N-terminal or lysine amino groups can be easily coupled to the protein using carbodimide or after converting the amino groups into activated NHS ester. Alternatively, one can also couple the phage through its SH groups to acrylamide that has been modified to include SH groups. The Versalinx slides are pre-modified with salicylhroxamic acid. The phage can be easily modified with phenyl boronic acid and a complex between the pehylboronic acid and salicylhorxamic acid is formed to immobilize the antibody on the glass slide. The technique is described by the manufacturer and is easy to use for coupling phage through the lysine amino group or the sulfhydril groups. Alternatively, the slides are coated with resins that impart a positive charge to the slides, which are commercially available. The phage or the bacteria can be arrayed directly on these slides using an automated slide spotter (Amersham Pharmacia Biotech). Several such automated slide spotters are commercially available.

Alternatively, the antibody can be excised from the phage or bacteria using proteolytic enzymes like stomolysin. The phage vectors and bacterial vectors have stromolysin or other protease sensitive sites built into the vector such that the antibody can be released by the protease treatment. The released antibody can then be purified on mini-protein A-Sepharose columns or antibody columns prior to arraying on the slides. The antibody can then be bound to slides that are coated with protein A. The phage that is treated with the protease is then arrayed on the Protein A slide and the remainder phage particles are washed off with a PBS-Tween 20 (0.1%) buffer. Arraying the antibodies as opposed to the phage or bacteria will reduce non-specific interaction with irrelevant proteins. Alternatively, the antibody is excised from the phage and separately purified and arrayed on slides.

Yet another method for arraying antibody on glass slides is to coat the slides with protein-A. Protein A binds IgG. The antibody displayed by the phage or bacteria are released by appropriate proteases (depending on the protease site engineered into the vector) and the mixture can then be directly spotted on the Protein-A coated slides. The antibody will specifically bind to the protein A and the all the remaining phage or bacterial proteins can be easily washed off with PBS-Tween. This method reduces the complexity of the operation.

Yet another way to immobilize the antibody is to engineer the cDNA that codes for 5-6 histidine moieties contiguously with the amino terminal or the carboxy terminal of the antibody or the Fab fragment. Stomolysin or similar protease sites are engineered on either end of the antibody-histidine tag fusion protein for easy excision of the antibody such that the antibody or Fab fragment will carry the histidine tag. The histidine residues can be utilized to bind the antibody or Fab to slides that have nickel immobilized on its surface. Published procedures for immobilizing nickel to glass slides or other supports are available.

Generally 10,000-50,000 separate phage or antibodies can be arrayed on one slide without loosing resolution during scanning. Higher densities of plating often reduce resolution.

Replicates of the antibody arrays are made and used for screening. The microarrays are stored at 4° C. until further use.

Labeling of Proteins:

In order to detect the binding of proteins to the antibody on the array a detection tag needs to be used. The commonly used detection tags are fluorescent or radioactive tags. CyDye, a fluorescent tag manufactured by Amersham Pharmacia is a good product for this purpose. The tag as activated NHS-esters and readily reacts with the carboxyl end of proteins. They also prevent cross-linking of proteins. Several other commercial dyes and activated esters of these dyes are available. Another method-is to radioiodinate the proteins in presence of chloramine T.

Prevention of Protein Aggregation or Non-Specific Protein-Protein Interactions:

It is important that the proteins remain as individual molecules and that they do not clump together (except when functionally it exists as dimmers or multimers or are linked to each other through disulfide bridges or exists are a multimeric protein unit as in the case of RNA polymerase). Often many unrelated proteins clump together through either hydrophobic or ionic interactions. This will cause multiple proteins to bind to the same antibody. We have devised a novel method to overcome this problem.

If there is significant non-specific clumping of proteins, the proteins are first treated with a protease that cleaves the protein infrequently like stromolysin or cathepsin D. This will release large fragments of the protein, which still retain their antigenicity, but are freed of protein-protein interactions. The aggregated fragments of the proteins are removed by either filtration or ultracentrifugation or by size exclusion chromatography. The free fragments are then labeled with the tag. Another efficient method of limited and specific cleavage of proteins is by treating the mixture with CNBr.

If this particular path is chosen for the preparation of protein samples, then the same protease-digested peptides must be used for panning selection of the phage to prevent contamination by phage displaying antibodies that recognize only the full-length protein or protein fragments that have been removed. Generally the more hydrophilic fragments tend to be free of protein-protein interactions and stay as free molecules.

Screening:

In order to achieve better representation of antibodies against as many proteins as possible, it is necessary to use sufficient microarrays that represent at least 0.5-1 million antibodies, Fab fragment or phage. The number of separate antibody or phage required could be as high as 10 million. It is certain that many antibodies will be represented multiple times on the array panel, but it is far more important to have a better representation of antibodies against diverse proteins and their modified forms. At times, far higher number of antibodies would be necessary to be used to get adequate representation.

There are several uses for these microarrays.

1. Comparison of the level of individual proteins between a normal and a disease tissue: Antibody microarry can be used to identify proteins that are differentially expressed in the normal and disease tissue. For this particular use, duplicate microarrays are used. One set of microarray will be incubated with labeled proteins from the normal tissue or cells and the duplicate set with labeled proteins from the disease tissue or cells. Proteins that are differentially expressed between a normal and disease tissue may be targets for therapeutic intervention or may be markers for the disease, but such inferences would require further validation of the targets.

2. Determination of the level of proteins from single tissue or cells: In this case only one set of microarry slide is used.

Overcoming Antigen Overload: Since the arrays are plated with the same amount of antibody at each position, it is conceivable that certain proteins that are present in high concentration in the cell or tissue may overload the antibody and the results obtained would not be representative. There are two solutions to this problem. Either one can increase the concentration of antibody that is spotted. Alternatively, antibodies can be coated at increasing concentration on separate slides so that saturation of any of the antibody can be easily monitored. If any protein saturates the antibody, there will be increased binding of protein to the antibody spotted at the higher concentration.

Protein Binding and Washing: The labeled proteins are incubated with the antibody in an incubation chamber for 30 min. to 2 hours. The slides are then washed with PBS containing 0.1% Tween 20 or other detergents and then scanned using a scanner depending on the label used. The entire process can be automated using an automated microarray washer similar to the one manufactured by Amersham-Pharmacia.

Scanning: If a fluorescent label is used, the microarrays are scanned in a fluorescence scanner. If the proteins are radioidodinated, a gamma ray scanner is used. The intensity of the spots is compared using commercial software similar to the one manufactured by Amersham-Pharmacia and compared.

Amplification of Signal: The sensitivity of the signal can be amplified by using standard amplification techniques. For example, the proteins can be biotinylated using a NHS-biotin ester, slides washed, then incubated with streptavidin conjugated to multiple molecules of horseradish peroxidase or alkaline phsophatase, washed, and the slides incubated with the substrates for the respective enzymes. Alternatively, the proteins can be conjugated to digoxigenin and the protein-digoxigenin conjugate detected using an anti-digoxigenin antibody conjugated to alkaline phosphatase or horseradish peroxidase.

Identification of Protein:

Identity of the proteins of interest can be determined by a variety of methods. In the preferred embodiment, adequate amount of the proteins of interest is purified from the protein extract from the tissue or cells. This can be accomplished by conjugating the antibody against the protein of interest to a matrix like Sepharose by published methods to generate an immunoaffinity column, the protein is purified by immunoaffinity chromatography, and the identity of the purified protein is determined. Alternatively, the identity of the antigen antibody conjugate that is fixed on the microarray itself could be conceivably determined.

Identity of the purified or antibody immobilized protein can be determined by a variety of methods. The protein is digested with proteolytic enzymes and a molecular weight fingerprint of the fragments can be identified by Matrix Assisted Laser Desorption Ionization—Time of Flight (MALDI-TOF) spectrometry and compared with protein digest patterns of proteins available with protein data banks like Swiss Prot. MALDI-TOF also gives the sequence information of small peptide fragments. The sequence identity of the peptide fragments after their separation by chromatography can be determined by electrospray spectrometry and Edman automated sequencing.

Key Steps of AMP Technology:

1. Extract proteins from normal and disease tissue

2. Remove major structural proteins (like actin, histones) by affinity chromatography

3. Inject proteins into mice, boost several times

4. Extract lymphoid mRNA, reverse transcribe immunoglobulin heavy chain (V_{H}) and light chain variable domain (V_{L}) repertoires

5. Display total V_{H} and L_{H} on M13 helper phage (or in bacteria or other hosts)

6. Subject phage to competitive deselection of dominant epitopes

7. Pan phage library 1-3 times (in certain instances more) on immobilized protein (or peptide fragments) used for immunization in order to enrich phage-expressing antibody to proteins present in target tissue (immunodominat epitope deselection).

8. Expand each phage plaque in microtiter plates using automated phage picker.

9. Array phage displaying different antibody microarrayed on glass or other chips using a fully-automated spotter

10. Bind fluorescent-labeled or radiolabled proteins extracted from diseased tissue and normal tissue to antibody microarray chip. The proteins may have to be treated with proteases to release large fragments and such fragments are separated by centrifugation prior to labeling and binding to reduce non-specific binding to the array due to clumping of proteins. The chips are washed using a fully-automated chip washing machine

11. Chip scanned using a fully-automated or manual scanner

12. Proteins that are differently expressed in diseased and normal tissue are identified using a commercial software program.

13. Proteins of interest are re-selected on a larger scale using the specific monoclonal antibody that recognizes the protein.

14. The peptide sequence fingerprinting of the protein performed using MALDI-TOF or using automated peptide sequencing machine

15. The proteins are analyzed using various protein and gene database (bioinformatics)

### SPECIFIC EXAMPLES

### Example 1

Removal of Major Structural and Other Proteins:

The tissues or cells are solublized using a variety of published techniques (Methods in Enzymol., Vol, 31). In a nutshell, fresh tissue is removed and cut into small pieces (2 mm) and washed with ice-cold PBS and then ground using a Dounce homogenizer or a Potter-Elvejhem homogenizer or a Polytron homogenizer in presence of a detergent like 1% Triton X-100 or 1% NP 40 or 2% CHAPS or other commonly used detergents. For solublizing cultured cells, a solubilizing agent called M-PER Pierce Chemical Company, Rockford, Ill.)or similar reagents are used. 25 volumes of ice-cold M-PER reagent containing protease inhibitors were added and the cells were homogenized using a Dounce homogenizer (20 strokes). The homogenate is centrifuged at 100,000 g for 1 hour and the supernatant removed. The solublized proteins present in the supernatant were used for further steps. In certain cases, the 0.25M sucrose was included (for brain tissue 0.32M).

When serum samples were used, no solublization reagents or detergents were used. The sample was used directly for the next step.

When only cytosolic proteins were used, the cells were lysed by either hypotonic shock in conjunction with Dounce homogenization in phosphate buffer, pH 7.2, containing 10 mM NaCl. The cytosolic fraction was separated from the rest of the cell fractions by centrifuging at 100,000 g for 1 hand recovering the supernatant.

All solubilization or extraction buffers contained 0.1M PMSF, 0.2 mM EDTA and 0.8 mM benzamidine and in some cases 10 ug/ml of Leupeptin and/or aprotinin was also included to prevent proteolytic degradation (if the latter were added, they have to removed by immunoaffinity chromatography prior to immunization). The entire process was done between 0-4° C. The extracted proteins are processed as immediately to prevent any proteolytic degradation or snap-frozen for later use.

Preparation of Immunoaffinity Column: Immunoaffinity resins to remove major structural and other major proteins were made as follows. Monoclonal or affinity-purified polyclonal antibodies against the various major proteins are commercially available. The antibodies are conjugated to NH-activated Sepharose (Amerhsam-Pharmacia) or similar supports according to the methods prescribed by the manufacturer. The antibody concentration was maintained at approximately 1 mg/ml of matrix (Sepharose), although this was not very critical.

Several immunoaffinity matrix representing the various structural proteins were mixed according to their approximate proportion in the cells to make a mixed bed resin. Examples of proteins that are removed include histone proteins, ribosomal proteins, polymerases, cytoskeletal components, albumin etc. The columns were equilibrated with Tris-buffered saline.

The protein fraction was loaded onto these columns at a slow rate (one column volume 20 min.) at 4° C. and the fractions that did not bind to the protein were collected and used for immunization. The same method was also applied to make protein fractions for screening, if one was not interested in studying the structural proteins. When the profile of expression of structural proteins also needed to be studied, then the antibodies against these proteins were also included in the microarray.

Sometimes it is necessary to reduce the complexity (number of different proteins in the sample) prior to injection into mice. This can be accomplished by different chromatographic or electrophoretic methods or other commonly used methods of fractionation. When the sample is constituted of digested peptide fragments, they are separated into multiple fractions (5-50) on a C-3 or C-18 or similar reverse phase HPLC column. Whole proteins are separated on an anion exchange chromatographic column (Pharmacia Amersham FPLC column) or a size exclusion chromatographic column. However, any of the several methods for fractionating proteins can be used alone or in combination to separate the proteins into various fractions prior to injecting into mice, if the antibody library efficiency is not adequate in the absence of such fractionation.

### Example 2

Blocking Immunodominant Epitopes by Competitive Deselection

The protocol is a modification of a standard panning protocol, except that the phage library is first preabsorbed on the antigen of interest to remove phage that react with the immunodominant epitope. The unbound phage are then incubated a second time with antigen, and eluted and amplified as per normal protocols.

1. Materials used

1. Antibody phage library freshly amplified according to standard protocols (see Note 1).

2. 0.1M carbonate buffer pH 8.6: for 1 liter, dissolve 8.4 g of NaHCO₃ in water, adjust pH to 8.6, filter and store at 4° C.

3. PBS; PBS/0.5% Tween 20; PBS/1% bovine serum albumin (BSA); 2M Tris base.

4. Elution Buffer: per 200 ml, add 1.6 ml of 12M HCl to water, adjust to pH 2.2 with solid glycine. Autoclave and store at room temperature.

5. VCS M13 helper phage; E. coli strain XL1-Blue.

6. Tetracycline stock: 5 mg/ml in ethanol; carbenicillin stock: 50 mg/ml in water; kanamycin stock: 10 mg/ml in water; store all antibiotic stocks at −20° C.

7. Superbroth medium (SB); SB with tetracycline (10 μg/ml final, concentration); SB/carbenicillin (20 μg/ml in low carbenicillin SB; 50 μg/ml in high carbenicillin SB); LB agar/carb plates (100 μg/ml carbenicillin).

8. PEG/NaCl:PEG-8000 20% w/v; NaCl 2.5M, autoclave and store at room temperature.

9. ELISA Plates (half area, high affinity binding: Costar Cat. No.3690); Oak Ridge centrifuge tubes (Sigma, Saint Louis, Miss.) or immunotubes

2. Procedure

1. Coat ELISA plates or immunotubes with the appropriate amount of antigen in each well (see Note 2). Coat both the adsorption and panning plate at the same time. For the adsorption plate coat at least 10 wells for each phage selection. For the first round of panning coat 4 wells; for the following rounds coat 2 wells per phage selection.

2. Incubate the sealed plate at 4° C. overnight.

3. Wash both plates 5 times with distilled water (100 μl/well), then blot dry on paper towel. Block both plates with 150 ul/well of PBS/1% BSA 2 hours at 37° C. Do not let the plates dry out (see Note 4).

4. At the same time inoculate a single fresh colony of E. coli XL1-Blue into 15 ml SB+tetracycline in a 50 ml tube. Grow at 37° C in a rotatory shaker. Start the culture in time to have bacteria ready for infection of step 9 (see Note 5).

5. Remove the block solution from the wells of the adsorption plate with a pipette and add 20 ul (>10¹⁰ cfu) of fresh library phage suspension to each of the ten wells. Phage is resuspended as in step 16. Seal the plate and incubate at 37° C. for 2 h.

6. Carefully and gently remove 15 ul of phage from each well of the adsorption plate and combine in a 0.5 ml microfuge tube and keep on ice. Remove the blocking solution from the panning plate and immediately add 50-70 ul of adsorbed phage to each well (25-35 μl in the first round of panning using 4 wells) Seal the plate and incubate for an additional 2 h at 37° C.

7. Wash the wells or tube 10× with PBS/0.5% Tween 20 (100 μl/well), leave for 5 min then discard washing solution. Use barrier tips to avoid contamination.

8. Elute the phages bearing specific antibodies by adding 50 ul of elution buffer per well or 1-5 ml for the immuotube depending on the size of the tube and incubating at room temperature for 3 min. Remove the elution buffer into a microfuge tube and neutralize immediately by adding 3 ul of 2M Tris per 50 ul of elution buffer for the ELISA plates and 1 mil for immunotubes.

9. Add the eluted phage to 2 ml of exponential growth phase E. coli XL1 Blue (density O.D.₆₀₀=0.6) and incubate in a rotatory shaker (230 rpm) for 15 min at 37° C.

10. Add 10 ml of prewarmed SB (low carbenicillin and tetracycline) to the 2 ml of infected cells. Plate 10, 1 and 0.1 ul of the infected cell suspension on LB/carb plates and incubate overnight at 37° C. Calculate the approximate number of eluted phages from the number of colonies. Incubate the remaining cell suspension for 1 h at 37° C. in a shaker.

11. Add 100 ml of pre-warmed SB (high carbenicillin and tetracycline) and incubate for 1 h at 37° C. in a shaker.

12. Add 10¹² pfu of helper phage VCSM13. Incubate at 37° C. in a shaker for a further 2 h.

13. Add kanamycin to a final concentration of 70 ug/ml, and incubate the culture overnight at 30° C. in a shaker.

14. Centrifuge the cultures at 2,000 g at room temperature in 50 ml tubes in a bench centrifuge. Precipitate the phage from the resulting supernatant by adding 7 ml of PEG/NaCl solution to 30 ml of supernatant in Oak Ridge tubes. Incubate on ice for 30 min.

15. Centrifuge for 20′ at 15,000 g at 4° C. and discard the supernatant. Let the tubes dry upside down on paper towel for 2-4 min.

16. Carefully resuspend the phage pellet in 1 ml PBS/1% BSA per tube. Be careful to also resuspend the pellet that usually forms on the wall of the tube. Transfer the suspension to a microfuge tube.

17. Mix the tube by inverting several times (do not vortex). Centrifuge at 10,000 g in a microfuge at 4° C. for 15 min then transfer the supernatant into a clean Eppendorf tube.

18. Use this phage suspension in subsequent rounds of panning (see Notes 6 and 7). It is advisable to use four wells in the first round of panning and two for the following rounds. Phage suspension from round showing enrichment can also be used to infect cells for the production of soluble Fab (Burioni et al.1998).

3. Notes on Procedure

1. The phage library or subsequently selected phage needs to be freshly amplified for each panning cycle. Although phage molecules themselves are very stable and can be stored for years at −70° C. without losing infectivity, displayed antibody molecules on the surface are not stable. Panning of a stored phage preparation can yield unpredictable results.

2. As a general rule dilute the antigen at a concentration 5 times greater than that used in ELISA for detection of antibodies. If this ELISA concentration is not known use 500 ng/well of antigen for panning, and 100 ng/well for ELISA. This concentration is usually suitable for the isolation of antibody-bearing phages. The volume in which the antigen is added can range from 25 to 50 ul. Optimal conditions for binding including temperature of binding and coating buffer need to be determined experimentally for each individual antigen. Most proteins bind well in PBS or in 0.1M carbonate buffer.

3. Check that the washing conditions used in panning do not detach bound antigen from the surface of the plate. To exclude this possibility coat an ELISA plate with antigen, wash using the same conditions used for panning, and then use the plate for an ELISA using a antibody known to react with antigen.

4. Proper blocking of the wells is crucial. The procedure has to be performed simultaneously for both the adsorption and panning plate. Do not let the wells dry out at any stage.

5. Infection of bacteria is a critical step. It is important that the optical density (OD) of the E. coli culture is approximately that indicated (i.e. exponential growth) in order to obtain maximal infection. Do not dilute the bacterial culture to obtain the correct OD but schedule the time of inoculation of the culture appropriately.

6. Do not reuse the plates or immuotubes. Use a fresh plate for each round of panning, and a different one for adsorption each time. The best results are obtained using plates freshly coated with antigen.

7. It is a crucial point to obtain a suitable phage preparation for performing this procedure. Low phage yield (<10¹¹ cfu/ml) usually does not allow a successful subtraction and cloning. However it is obviously impossible to know the titer of a phage preparation at the starting point, as it would take at least one day to determine it and the phage has to be used freshly prepared. We usually rely on the evaluation of the growth of bacteria as seen before centrifugation. A poor bacterial growth prompts for a restart.

8. Subtraction is not as efficient as selection. For this reason it is necessary to use ten wells for absorption. In case of over-representation of clones this number can be doubled, trying to keep to a minimum the total volume of phage. It should be noted that the first panning round is crucial for successful selection. Number of wells can be increased as well as incubation time. Some antigens require incubation at 4° C. overnight, even if this is rarely needed. In these cases subtract for 5 hours at 4° C. using the same procedure. Do not incubate more than overnight

9. It is not advisable to perform more than 3 rounds of panning. After this point a dominant (and often unwanted) clone will emerge.

### Example 3

Proteolysis of Target Protein to Prevent Aggregation.

It is essential that any aggregates of target proteins or heteromeric protein-protein interactions are disrupted prior to binding to the microarray. Such interactions are difficult to break up hence we have devised a method to overcome the problem. Presence of proteins that exist normally as dimmers or multimers or proteins that are functionally multimeric does not pose a problem with the method. The target protein mixture is treated with a protease like stromolysin or Catepsin D, which has fewer proteolytic sites on proteins. Essentially, the target proteins are incubated with 10 ug/ml of the protease and incubated at 20-37° C. for 30 minutes in Tris-HCl -or phosphate buffer (at the optimal pH of the protease) containing 150 mM NaCl (to break up potential ionic interactions) The proteases release large fragments of the proteins. The hydrophobic portions of the proteins usually tend to aggregate in aqueous media, while the hydrophilic pieces generally remain non-aggregated and free. Another method for cleaving large proteins into fragments is through the use of CNBr according to published methods. The aggregated proteins are removed by passing them though a size exclusion columns. The breakthough fractions containing the aggregated proteins fragments are discarded, while the dispersed hydrophilic molecules are eluted and concentrated prior to use. Alternatively the peptide fraction is separated from the aggregated protein fraction by passing it through an Amicon ultrafiltration devise with a molecular weight cut-off of 10,000-25,000 as described by the manufacturer. The ultrafiltrate is collected, concentrated by passing it through an anion exchange column at pH 8.0 and eluted by lowering the pH or increasing the salt concentration. One could also use a variety of other published methods for concentrating the peptides

Alternatively, the digested proteins are centrifuged at 100,000 g for 2 hours to sediment the aggregated fragments. The supernatant is collected and concentrated by methods mentioned above prior to conjugating with a detection label.

### Example 4

Labeling of Proteins and Peptides:

A variety of commercially available labeling reagents which includes fluorescent dyes, radiolabel or chemiluminescent label can be used for labeling proteins. In the preferred embodiment, CyDye (Amersham Pharmacia Biotech, N.J., USA) is used as described by the manufacturer. In a nutshell, the protein or peptide samples are incubated with the mono-reactive dye and the unreacted dye is removed. The dye reacts with the amino groups present in the protein or peptide.

### Example 5

Preparation of Antibody Microarrays:

Micro arraying of antibodies or Fab fragments are done as follows. Phage suspension at an appropriate concentration are used to infect E. Coli as described above in the procedure section. Infected cells are plated (after incubation) in LB/agar plates containing penicillin and tetracycline. Bacterial clones growing in ampicillin are containing the antibody genes bearing phagemids. Bacterial colonies are picked using an automated picker and grown at 37° C. in 96 well plates in SB (ampicillin-tetracycline). In the case of the use of antibodies for array construction, infected bacteria are of the non suppressor strain and will produce soluble Fabs. In this case after 5 hours of growth IPTG at a concentration of 1 mM is added and cells are cultured for further 15 hours at 30° C. The plates are spun down and the supernatant removed using an automated liquid handling station. 50 ul aliquot of the supernatant (containing depending on the procedure phage or free phagemid, that can be converted to phage upon transformation and helper phage infection of host cells) is removed and stored at −20° C. as glycerol stock for future use after properly cataloguing it. If the antibody or Fab fragments displayed on the surface of the page has proteolytic cleavage sequences engineered on the flaking regions, the phage is treated (if needed) with the proteolytic enzyme to release the Fab fragment or the antibodies.

For antibody array construction the digest is passed over immunoaffinity column of mouse anti-Fab antibody or single chain antibody coupled to Sephacryl. The columns are in the same 96-well plate format for ease of use. Alternatively, Protein-A-Sepharcryl column can be used. The antibody or Fab fragment is eluted from the column and collected in tubes in the 96-well plate format and stored in cold until arrayed on slides. The arraying performed as soon as possible.

When the bacteria is used for displaying the Fab fragments or antibody, the same procedure is used except that the colonies are grown overnight in LB in 96 well plates (Quiagen), a portion of the bacteria removed and stored as glycerol stock at −90C and the remaining bacteria spun down using centrifuge with a 96-well plate adapter, the pellet washed two times in M8 salt solution and resuspended in the same solution and the Fab fragments or the antibody excised with the proteases. After excision of the antibody, the bacteria are spun down, the supernatant containing the antibody or Fab are removed and purified as above and stored in 4C.

One could also spot the phage directly on the microarray. In this case, if the procedure is intended to create phage microarrays, bacterial cells are grown for 1 hour at 37° C. and after this step 5 μl of a suspension of helper phage (containing >10¹² pfu/ml) are added to the wells. After 2 hrs of incubation at 37° C. kanamicine is added to a final concentraion of 70 μg/ml. Cells are grown for further 15 hrs at 30° C. In this case the supernatant is containing phage.

Preparation of antibody microarray: Prior to spotting the antibodies, an analytical test is performed to determine the efficiency of the antibody library. This step is done prior to the harvesting of colonies described above. Typically sufficiently large number of phage clones (between 0.5-10 million) is suspended in PBS/BSA1% and panned against very low abundance proteins or peptides. Presence of protein-specific clones is then determined by conventional methods. These proteins and peptides are chosen based on their level of expression in the particular tissue or cell that is used. Alternatively, one could spike the immunization mixture used for immunizing the mouse with protein from a different source at a very low concentration (e.g. 1 part in 200,000 or 1 part in million) and screened for the presence of antibody against this protein.

Alternatively the screening is done by transferring the phage to nylon membranes and screening for the presence of the antibody using the respective antigens that are conjugated to horseradish peroxidase or alkaline phosphatase (Maniatis, 1991). The efficiency of the library is represented by the number of positive plaques in one million phage or bacterial clones. When microarrying, care is taken to make sure that sufficient number of phage or bacteria are chosen that these low abundant proteins are represented 2-4 times in the library.

Once the efficiency of the library has been determined and the number of antibody, Fab, phage to be microarryed has been determined, the samples are processed as described above and they are microarryed on an appropriate glass slide coated with a medium that binds proteins efficiently like polystyrene. It is more efficient to use slides that are coated with agents that form strong bonds with proteins. In the preferred embodiment, Versalinx slides (Prolinx, Bothell, Wash.) are used. Versalinx glass slides are coated with pheylyboronic acid and all non-specific protein binding sites are preblocked (an additional blocking with 0.1% BSA can be done after arraying, if needed). The antibody or Fab or phage is treated with salicylhydroxamic acid (as described by the manufacturer) and then spotted on the slides. The salicylhydoxamic acid moieties on the antibody specifically bind to the pheylboronic acid moiety thereby immobilizing the antibody. One could use any technique that immobilizes proteins on slides for the purpose of arraying.

Arraying of antibodies is performed using an automated microarray spotter similar to the one manufactured by Pharmacia Amersham, (Piscataway, N.J.). Replicates of the slides are made and stored at 4C until use. Usually 0.5-10 million antibody, Fab or phage particles are spotted depending on the efficiency of the antibody library as well as the abundance of the proteins of interest in the library. Alternatively, the slide can be coated with anti-phage or anti-Fab antibodies, or with proteins able to bind to the light chain of the Fab.

The fluorescent-tagged peptide fragments or protein from disease and normal tissue are incubated in separate identical slides in PBS buffer and incubated for 1-10 hours. The binding is done in an automated slide processor (Pharmacia Amersham) or similar equipment. The slides are washed with PBS buffer, slides removed and scanned using a fluorescence scanner (Pharmacia Amersham) or similar and the fluorescence acquired and analyzed using a Gen3DB software (Pharmacia Amersham) or similar software. Identical spots in duplicate slides which has significant difference in binding of the protein or peptide as determined by the difference in intensity of the fluorescence are potential protein targets of interest and are further evaluated.

1. A method for determining the level of known and unknown proteins in a tissue or cell, comprising: extracting the proteins from the target tissue or cell and using the proteins to generate an immune response in an appropriate animal host; cloning the antibody Fab fragment or the V_{H} and V_{L} chains or larger fragments of the antibody in phage such that the antibody is displayed on the surface; selecting the antibodies against the injected antigens by limited panning; isolating individual clones of phage; expanding the clones and spotting them on microarrays in sufficient number so that all or most the antibodies against the various proteins are represented in the microarray; allowing labeled protein from the target tissue or cell to bind to the antibodies on the microarray; and monitoring the level of the protein by scanning in a fluorescence reader machine, or a radioactive counter, or luminometer depending on the label.

2. The method of claim 1 wherein the host is selected from the group consisting of:
a) a transgenic animal that carry human V genes or other human immunoglobulin components so as to produce largerly human type antibodies; and
b) a mammal.

3. The method of claim 1 for use to compare the level of expression of proteins between a diseased and normal tissue or cell.

4. The method of claim 1 wherein the antibody is selected from a library of phage expressing an antibody and displaying a random sequence representing the variable sequences by the method of in-vitro selection.

5. The method of claim 1 wherein monoclonal antibody fragments against non-immunodominant antigenic determinants that are contained in the library phage display immunoselection have been obtained once the library of antibodies is first preabsorbed on the antigen of interest to remove antibody that react with the immunodominant epitopes and the unbound phage is then panned again with the same antigen.

6. The method of claim 1 wherein monoclonal antibody fragments against non immunodominant proteins are obtained from the library by panning against antigen(s) of interest after preabsorbing the library with immunodominant proteins contained in cells lysate.

7. The method of claim 1 wherein monoclonal antibody specific for proteins of diseased cells are obtained from the library by panning against diseased cells lysate after preabsorbing the library with proteins from healthy (related or unrelated) cells or with similar cells lacking protein(s) when compared to the target cell.

8. The method of claim 1 wherein the phage library bears antibody fragments from humans infected with pathogens or have autoimmune disease.

9. The method of claim 1 wherein proteins in abundance in cells, structural proteins and immunodominant proteins are removed by the method of immunoaffinity chromatography.

10. The method of claim 1 wherein the extracted proteins from target tissue or cells are treated with proteases or CNBr so as to release fragments which do not aggregate or interact with each other, the aggregated fragments removed by methods including, but not limited to size-exclusion chromatography, ion-exchange chromatography, ultracentrifugation and the non-aggregated peptides are further used for screening microarrays.

11. The method of claim 1 or 10 wherein the protein or peptides are conjugated to biotin, incubation with streptavidin that is conjugated with horse radish peroxidase or alkaline phosphatase or similar enzymes and detecting the presence of the peptide by incubation with the respective substrates for the enzymes.

12. The method of claim 1 or 8 wherein the protein or peptides are conjugated with digoxigenin, incubation with an antibody against digoxigenin that is conjugated with horse radish peroxidase or alkaline phosphatase or similar enzymes and detecting the presence of the peptide by incubation with the respective substrates for the enzymes.

13. The method of claim 1 wherein the vector is a phagemid, plasmid, lambda, filamentous phage, bacteria, yeast or other unicellular organisms.
